(19) United States
(12) Patent Application Publication
(10) Pub. No.: US 2021/0393157 A1
(43) Pub. Date: Dec. 23, 2021

(54) SYSTEMS AND METHODS FOR MULTI-POINT BIOIMPEDANCE MEASUREMENTS

(71) Applicant: AURA Devices, Inc., Wilmington, DE (US)

(72) Inventors: Stanislav Gorbunov, Moscow (RU); Igor Dorokhin, Saratov (RU); Andrey Boev, Stariy Oskol (RU); Dmitrii Davydov, Lublin (PL)

(21) Appl. No.: 16/907,587

(22) Filed: Jun. 22, 2020

(51) Int. Cl. A61B 5/053; A61B 5/00; G01G 19/50

(52) U.S. Cl. CPC A61B 5/053; A61B 5/6825; A61B 5/6831; A61B 5/6887; A61B 2562/04; G01G 19/50; A61B 2560/0475; A61B 2562/222; A61B 2562/0209; A61B 5/0004

(57) ABSTRACT

Systems and methods for performing combined inter-device multi-point bioimpedance measurements are provided. An example system includes a first device and a second device. Each device includes voltage electrodes, current electrode, controller, and connector for electrically coupling the electrodes of each other by a wire. The controller the first device or the second device can receive a configuration. The controller may select, based on the configuration, a first subset of current electrodes from a set including the current electrodes of the first device and the current electrodes of the second device and a second subset of voltage electrodes from a set including the voltage electrodes of the first device and the voltage electrodes of the second device. The controller can provide an electrical current to electrodes of the first subset and measure voltages from the electrodes of the second subset to determine bioimpedance parameters of the human body.

### TECHNICAL FIELD

The present disclosure relates generally to health monitoring and, more particularly, to systems and methods for multi-point bioimpedance measurements.

### BACKGROUND

Bioimpedance measurements and bioimpedance analysis can be used for estimating human body composition, and, more specifically, for determining amounts of fat mass and fat free mass of a human body, a body cell mass, and a total body water including extracellular fluid and intracellular fluid. Therefore, bioimpedance measurements are widely used in monitoring of health status and disease prognosis. Performing continuous daily or weekly bioimpedance measurements in a convenient manner may be specifically useful for detecting early warnings concerning health issues in humans.

Existing wearable devices for bioimpedance measurement, such as smart wearable bracelets, are designed to be worn on arms or legs and enable measurement of bioimpedance either from the upper part of a human body or the lower part of the human body. The contribution of the middle part of the human body (torso) in value of bioimpedance is assumed to be low due to the relatively large section of the torso. However, knowledge of bioimpedance for the entire body can be useful for accurate estimates of body composition. Therefore, there is a need for methods for obtaining bioimpedance information related to the entire human body using wearable devices allowing an assessment of body composition with the same precision as clinical bioimpedance devices and smart scale capable of performing bioimpedance analysis.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Provided are systems and methods for combined inter-device multi-point bioimpedance measurements. Some embodiments of the present disclosure provide smart straps and smart scales that can be configured to perform combined inter-device multi-point bioimpedance measurements. Furthermore, the smart straps can be used by a user for segmental hand-to-hand bioimpedance measurements and for segmental leg-to-leg bioimpedance measurements. The smart straps and the smart watch can also be connected by a wire to perform up to eight-point segmental or whole-body obtained legs-to-hands bioimpedance measurements.

According to one example embodiment, a system for combined inter-device multi-point bioimpedance measurement is provided. The system may include a first device and a second device. The first device may include a first voltage electrode and a first current electrode configured to touch a first zone of a first segment of the body of a user. The first device may include a second voltage electrode and a second current electrode configured to touch a second zone of the first segment of the body of the user. The first device may include a first connector electrically wired to the first voltage electrode, the first current electrode, the second voltage electrode, and the second current electrode. The first device may include a first controller coupled to the first connector, the first voltage electrode, the first current electrode, the second voltage electrode, and the second current electrode. The second device may include a third voltage electrode and a third current electrode configured to touch a third zone of a second segment of the body of the user.

The second device may include a fourth voltage electrode and a fourth current electrode configured to touch a third zone of the second segment of the body of the user. The second device may include a second connector electrically wired to the third voltage electrode, the third current electrode, the fourth voltage electrode, and the fourth current electrode. The second device may include a second controller coupled to the second connector, the third voltage electrode, the third current electrode, the fourth voltage electrode, and the fourth current electrode. The system may include a wire configured to connect the first connector and the second connector electrically. The first controller can be configured to receive a configuration for measuring bioimpedance. The first controller can select, based on the configuration, a first subset of current electrodes from a first set including the first current electrode, the second current electrode, the third current electrode, and the fourth current electrode. The first controller can select, based on the configuration, a second subset of voltage electrodes from a second set including the first voltage electrode, the second voltage electrode, the third voltage electrode, and the fourth voltage electrode. The first controller can provide an electrical current to electrodes of the first subset and measure voltages from the electrodes of the second subset. The first controller may determine, based on the voltages, bioimpedance parameters of the body of the user.

The first device can be a smartband. In the smartband, the first voltage electrode and the first current electrode can be configured to touch skin of a first hand of the user and the second voltage electrode and the second current electrode are configured to be touched by skin of a second hand of the user.

The second device can be a smart scale. In the smart, the third voltage electrode and the third current electrode can be configured to touch skin of a first foot of the user and the fourth voltage electrode and the fourth current electrode are configured to touch skin of a second foot of the user.

The first subset includes at least one current electrode of the first device and the second subset includes at least one voltage electrode of the second device. Alternatively, the first subset includes at least one voltage electrode of the first device and the second subset includes at least one current electrode of the second device.

Providing electrical current can be restricted to at least one of first current electrode and the second current electrode and measuring the voltages can be restricted to at least one of the third voltage electrode and fourth voltage electrodes. Alternatively, providing electrical current can be restricted to at least one of the third current electrode and the fourth current electrode and measuring the voltages can be restricted to at least one of the first voltage electrode and second voltage electrodes.

The first device may include a communication unit. The first controller can be configured to receive, via the communication unit, the configuration from an external computational device. The first controller can send, via the communication unit, the bioimpedance parameters to the external computing device. The first controller can also send raw data of the measured voltages to the external computing device and the external computing device can analyze the raw data to determine the bioimpedance parameters. The communication unit can be a wireless communication unit.

The first device can be configured to store first historical data of bioimpedance measured exclusively with the first voltage electrode and the first current electrode and without connecting to the second device. The second device can be configured to store second historical data of bioimpedance measured exclusively with the third voltage electrode, the third current electrode, the fourth voltage electrode, and the fourth current electrode without connecting to the first device. Upon connecting with the wire, the first device and the second device can exchange the first historical data and the second historical data.

According to another example embodiment, a method for performing combined inter-device multi-point bioimpedance measurements is provided. The method includes providing a first device, wherein the first device includes a first voltage electrode and a first current electrode configured to touch a first zone of a first segment of the body of a user. The first device includes a second voltage electrode and a second current electrode configured to touch a second zone of the first segment of the body of the user. The first device includes a first connector electrically wired to the first voltage electrode, the first current electrode, the second voltage electrode, and the second current electrode. The first device includes a first controller coupled to the first connector, the first voltage electrode, the first current electrode, the second voltage electrode, and the second current electrode. The method includes providing a second device. The second device includes a third voltage electrode and a third current electrode configured to touch a third zone of a second segment of the body of the user. The second device includes a fourth voltage electrode and a fourth current electrode configured to touch a third zone of the second segment of the body of the user. The second device includes a second connector electrically wired to the third voltage electrode, the third current electrode, the fourth voltage electrode, and the fourth current electrode. The second device includes a second controller coupled to the second connector, the third voltage electrode, the third current electrode, the fourth voltage electrode, and the fourth current electrode. The method may include providing a wire configured to electrically connect the first connector and the second connector.

The method may also include receiving, by the first controller, a configuration for measuring a bioimpedance. The method may include selecting, by the first controller and based on the configuration, a first subset of current electrodes from a first set including the first current electrode, the second current electrode, the third current electrode, and the fourth current electrode. The method may also include selecting, by the first controller and based on the configuration, a second subset of voltage electrodes from a second set including the first voltage electrode, the second voltage electrode, the third voltage electrode, and the fourth voltage electrode. The method may include providing, by the first controller, an electrical current to electrodes of the first subset. The method may include measuring, by the first controller, voltages from the electrodes of the second subset. The method may include determining, by the first controller and based on the voltages, bioimpedance parameters of the whole body of the user.

Additional objects, advantages, and novel features will be set forth in part in the detailed description section of this disclosure, which follows, and in part will become apparent to those skilled in the art upon examination of this specification and the accompanying drawings or may be learned by production or operation of the example embodiments. The objects and advantages of the concepts may be realized and attained by means of the methodologies, instrumentalities, and combinations particularly pointed out in the appended claims.

### BRIEF DESCRIPTION OF THE DRAWINGS

Embodiments are illustrated by way of example and not limitation in the figures of the accompanying drawings, in which like references indicate similar elements and, in which:

FIG. 1 shows an example system for combined inter-device multi-point bioimpedance measurements, according to some embodiments of the present disclosure.

FIG. 2 is a block diagram of an example smartband for bioimpedance measurements, according to some example embodiments.

FIG. 3 is a block diagram of an example smart scale for bioimpedance measurements, according to some example embodiments.

FIG. 4 is a schematic diagram showing example configurations for combined inter-device multi-point bioimpedance measurements, according to various example embodiments.

FIG. 5 is a flow chart of example method for bioimpedance measurements, according to some example embodiments.

FIG. 6 shows a computing system that can be used to implement embodiments of the present disclosure.

### DETAILED DESCRIPTION

The following detailed description includes references to the accompanying drawings, which form a part of the detailed description. The drawings show illustrations in accordance with example embodiments. These example embodiments, which are also referred to herein as “examples,” are described in enough detail to enable those skilled in the art to practice the present subject matter. The embodiments can be combined, other embodiments can be utilized, or structural, logical, and electrical changes can be made without departing from the scope of what is claimed. The following detailed description is, therefore, not to be taken in a limiting sense, and the scope is defined by the appended claims and their equivalents.

The present disclosure provides methods and systems for combined inter-device multi-point bioimpedance measurements. Embodiments of the present disclosure allow for combining at least two different devices for combined inter-device bioimpedance measurements. The first device can be a smartband designed to be worn on a hand of a user. The smartband may include a function of performing segmental hand-to-hand bioimpedance measurements. The second device can be a smart scale having a function for segmental leg-to-leg bioimpedance measurements. Each of the devices can be used separately and in combination. Combining the first device and the second device can be carried out by electrically connecting the devices by a wire via a connector in one of the devices or connectors in both devices. A body of the smartband may include a specially designed connector to receive the wire. The wire can be rigidly connected to the smart scale and kept in an internal compartment of the smart scale when the devices are not used in combination.

The smartband and the smart scale each may have autonomic power supply and can be used to perform impedance measurements independently of each other. The smart scale can be used for both measurement of weight of the user and measurement of bioimpedance of the user. Both the smartband and the smart scale may process results of the bioimpedance measurements, store the results of the bioimpedance measurements, and exchange the results of the bioimpedance measurements between each other. The smartband and the smart scale may be wirelessly connected to a smartphone, or other personal computing device, and send the results of bioimpedance measurements to the smartphone or exchange the results of the bioimpedance measurement via the smartphone.

After being connected via the wire, the two devices can be used for performing combined multi-point segmental or whole-body leg-to-hand bioimpedance measurements. The results of the multi-point leg-to-hand bioimpedance measurements can be kept in a memory of the smartband and/or the smart scale and can be used as a reference value. The reference value can be used in analysis of the further bioimpedance data that can be obtained using only the smartband or only the smart scales to estimate a state of the body composition of the user and changes in the body composition.

According to one example embodiment, an example system for performing multi-point bioimpedance measurements may include a first device and a second device. The first device may include a first voltage electrode and a first current electrode configured to touch a first zone of a body of a user. The first device may include a second voltage electrode and a second current electrode configured to touch a second zone of the body of the user. The first device may include a first connector electrically wired to the first voltage electrode, the first current electrode, the second voltage electrode, and the second current electrode. The first device may include a first controller coupled to the first connector, the first voltage electrode, the first current electrode, the second voltage electrode, and the second current electrode. The second device may include a third voltage electrode and a third current electrode configured to touch a third zone of the body of the user. The second device may include a fourth voltage electrode and a fourth current electrode configured to touch a third zone of the body of the user. The second device may include a second connector electrically wired to the third voltage electrode, the third current electrode, the fourth voltage electrode, and the fourth current electrode. The second device may include a second controller coupled to the second connector, the third voltage electrode, the third current electrode, the fourth voltage electrode, and the fourth current electrode.

The system for performing combined inter-device multi-point bioimpedance measurements may include a wire configured to electrically connect the first connector and the second connector. The system may include an external computing device communicatively connected to the first device and the second device. At least one of the first controller and the second controller can be configured to receive, from the external computing device, a configuration for measuring bioimpedance. One of the first controller and the second controller can select, based on the configuration, a first subset of current electrodes from a first set including the first current electrode, the second current electrode, the third current electrode, and the fourth current electrode. One of the first controller and the second controller can select a second subset of voltage electrodes from a second set including the first voltage electrode, the second voltage electrode, the third voltage electrode, and the fourth voltage electrode. One of the first controller and the second controller can provide an electrical current to electrodes of the first subset. One of the first controller and the second controller can measure voltages from the electrodes of the second subset. Based on the voltages, one of the first controller and the second controller can determine bioimpedance parameters and send the bioimpedance parameters to the external computing device.

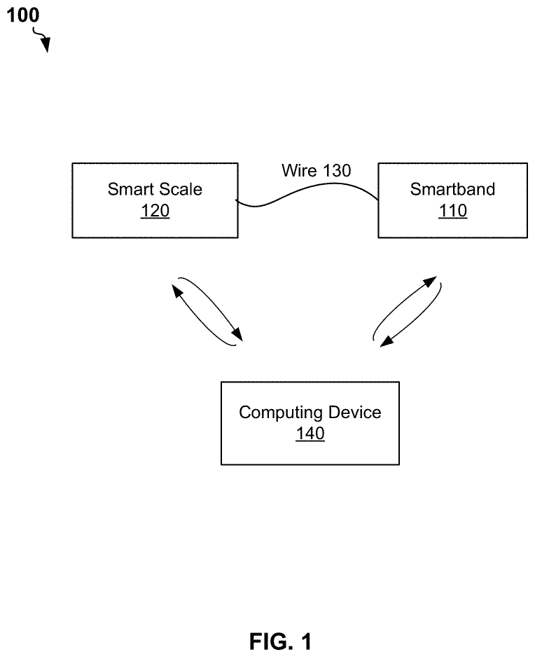

Referring now to the drawings, FIG. 1 shows an example system 100 for combined inter-device multi-point bioimpedance measurements, according to some embodiments of the present disclosure. The system 100 may include a smartband 110, smart scale 120, and a computing device 140. The smartband 110 may include a smart device wearable around a wrist of a user. Such a wearable device may include smart watch, smart bracelet, smart strap for smart watch, and the like. The smartband may be designed to measure hand-to-hand bioimpedance of the user.

The smart scale 120 may include an electronic device configured to measure a weight of the user when the user is standing on it. In addition to measuring the weight of the user, smart scale 120 may be designed to measure a leg-to-leg bioimpedance of the user.

The smartband 110 and smart scale 120 can be connected via a wire 130 via a connector of smartband 110 and a connector of smart scale 120. The wire 130 can electrically connect current electrodes and voltage electrodes of smart scale 120 to a bioimpedance measurement module of smartband 110 to allow whole body hand-to-leg bioimpedance measurements by applying an electrical current to current electrodes of the smart strap 110 and measuring voltages on voltage electrodes of smart scale 120. Alternatively, the whole-body hand-to-leg bioimpedance measurements can be carried out by applying electrical current to current electrodes of the smart scale 120 and measuring voltages on voltage electrodes of smartband 110.

In some embodiments, wire 130 can be removable from both smartband 110 and smart scale 120. In other embodiments, wire 130 may be permanently connected to smart scale 120. In these embodiments, the smart scale 120 may include an internal compartment for folding and storing wire 130. When a user of smartband 110 and smart scale 120 needs to perform combined inter-device multi-point hand-to-leg bioimpedance measurements, wire 130 can be unfolded from the internal compartment of smart scale 120 and connected to the smartband 110.

In some embodiments, smartband 110 and smart scale 120 can perform bioimpedance measurements independent of each other. Historical data on separate bioimpedance measurements can be stored locally on smartband 110 or smart scale 120. The smartband 110 and smart scale 120 can exchange historical data on separate bioimpedance measurements when they are connected via wire 130 to recalibrate and/or integrate two segmental bioimpedance measurements. In other embodiments, smartband 110 and smart scales 120 can be connected using a wireless connection. In these embodiments, the historical data on separate bioimpedance measurements can be exchanged wirelessly to recalibrate and/or integrate two segmental bioimpedance measurements.

The computing device 140 may include a personal computer (PC), a laptop, a smartphone, a tablet PC, a personal wearable device, and so forth. The computing device 140 can be configured to receive data from smartband 110 and smart scales 120. In some embodiments, the data may include results of a bioimpedance measurement performed by smartband 110 and smart scale 120. In other embodiments, the data may include raw sensor data, such as voltages from voltages electrodes of smartband 110 and smart scale 120 and electrical currents provided to current electrodes of smartband 110 and smart scale 120. The computing device 140 can perform combined inter-device multi-point analysis to determine multifrequency bioimpedance of the body of the user and body composition.

The computing device 140 may include an application allowing to send a configuration for separate segmental bioimpedance measurements or combined inter-device bioimpedance measurements to smartband 110 and/or smart scale 120. The configuration may include information regarding which current electrodes and voltages electrodes of smartband 110 and/or smart scale 120 should be used in bioimpedance measurements performed mutually by smartband 110 and smart scales 120.

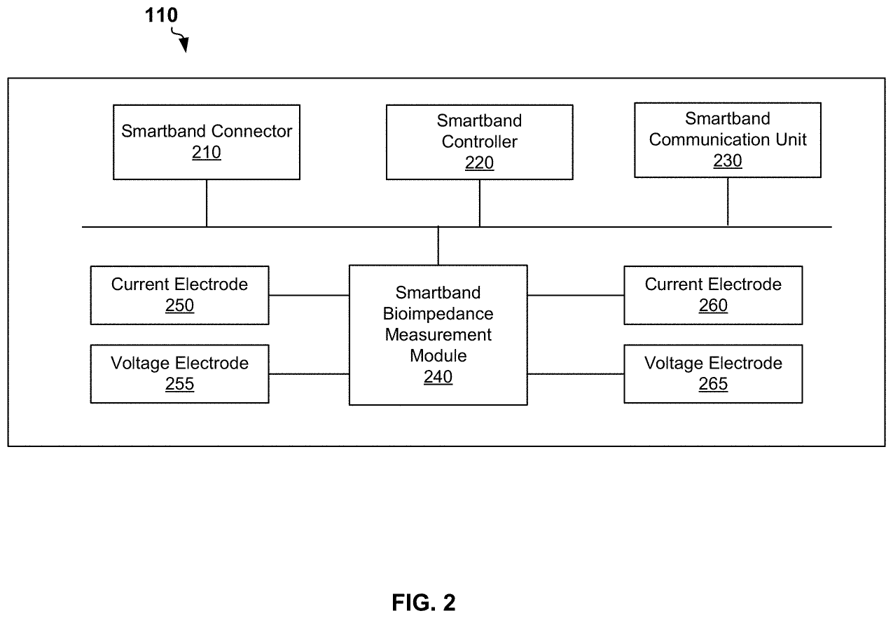

FIG. 2 is a block diagram of a smartband 110 for bioimpedance measurements, according to some example embodiments. The smartband 110 may include smartband connector 210, smartband controller 220, a smartband communication unit 230, a smartband bioimpedance measurement module 240, current electrodes 250 and 260, and voltage electrodes 255 and 265.

In some embodiments, current electrode 250 and voltage electrode 255 are located on an inner surface of smartband 110. When the user wears smartband 110 on a hand (at a wrist), current electrode 250 and voltage electrode 255 are in constant contact with skin of the hand of the user. Current electrode 260 and voltage electrode 265 can be located on an outer surface of the smartband 110. A user may place a palm or fingers of another hand to bring skin of another hand in contact with current electrode 260 and voltage electrode 265 during bioimpedance measurement.

The smartband bioimpedance measurement module 240 can be controlled by the smartband controller 220 to perform bioimpedance measurements which include providing electrical current to current electrodes 250 and 260 and sensing voltage difference at voltage electrodes 255 and 265.

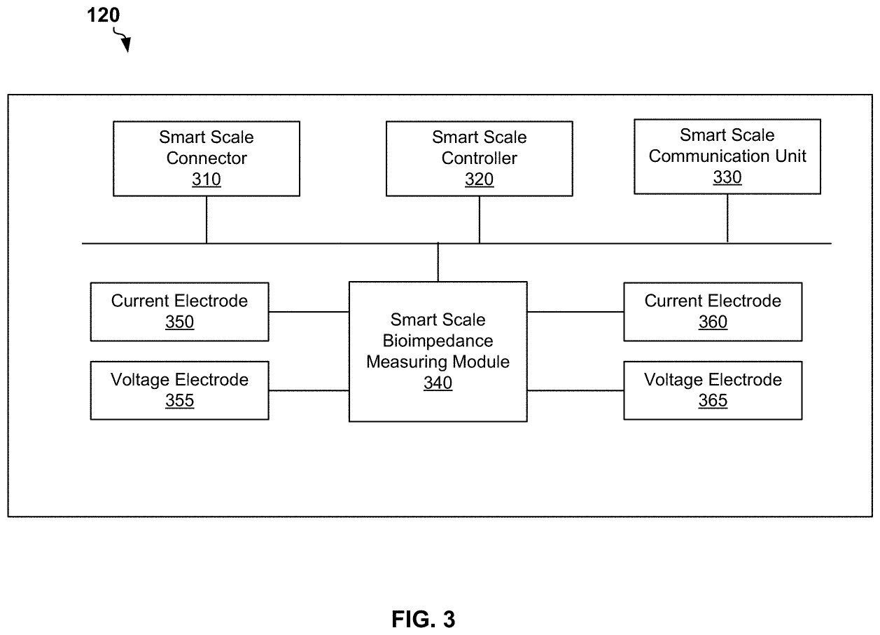

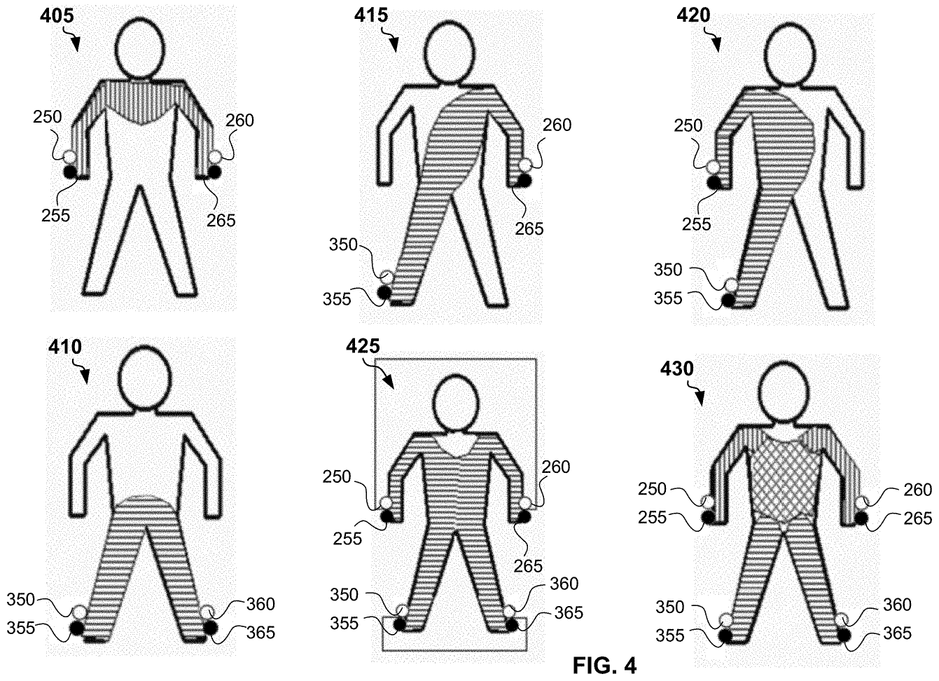

The smartband connector 210 can be configured to accept wire 130 and so electrically connect smartband bioimpedance measurement module 240 to the voltage electrodes and current electrodes of a smart scale 120. The smartband controller 220 can further configure smartband bioimpedance measurement module 240 to perform bioimpedance measurement using both current electrodes 250 and 260 and voltage electrodes 255 and 265 of the smartband 110 and current electrodes and voltage electrodes of the smart scale 120 (shown in FIG. 3).

FIG. 3 is a block diagram of a smart scale 120 for bioimpedance measurements, according to some example embodiments. The smart scale 120 may include a smart scale connector 310, a smart scale controller 320, a smart scale communication unit 330, a smart scale bioimpedance measurement module 340, current electrodes 350 and 360, and voltage electrodes 355 and 365.

In some embodiments, current electrode 350 and voltage electrode 355 are located on the left side of an upper surface of the smart scale 120 to allow the user stepping on them by the left foot. Current electrode 360 and voltage electrode 365 are located on the right side of the upper surface of the smart scale 120 to allow the user stepping on them by the right foot. When the user is standing on the smart scale, the current electrode 350 and the voltage electrode 355 are in contact with skin of the left foot of the user and current electrode 360 and voltage electrode 365 are in contact with skin of the right foot of the user.

The smart scale bioimpedance measurement module 340 can be controlled by the smart scale controller 320 to perform bioimpedance measurements, which include providing electrical current to current electrodes 350 and 360 and sensing a voltage difference at voltage electrodes 355 and 365.

The smart scale connector 310 can be configured to accept wire 130 and so electrically connect the smart scale bioimpedance measurement module 340 to voltage electrodes 255 and 265 and current electrodes 250 and 260 of smartband 110. The smart scale controller 320 can further configure the smart scale bioimpedance measurement module 340 to perform bioimpedance measurement using both current electrodes 350 and 360 and voltage electrodes 355 and 365 of the smart scale 130 and current electrodes 250 and 260 and voltage electrodes 255 and 265 of smartband 110 (shown in FIG. 2).

In some embodiments, the smart scale connector 310 may not be present in the smart scale. In these embodiments, wire 130 can be rigidly connected to the smart scale bioimpedance measurement module 340. The smartband bioimpedance measurement module 340 can be connected to voltage electrodes 255 and 265 and current electrodes 250 and 260 of smartband 110 by connecting wire 130 to the smartband connector 210 (shown in FIG. 2).

FIG. 4 is a schematic diagram showing example configurations 405, 410, 415, 420, 425, and 430 for combined inter-device multi-point bioimpedance measurements, according to various example embodiments. The configurations 405, 410, 415, 420, 425, and 430 may indicate which of the current electrodes and voltage electrodes of smartband 110 and smart scale 120 are to be used in bioimpedance measurements. The configurations can be received by either smartband controller 220 or smart scale controller 320 from, for example, computing device 140 (shown in FIG. 1). Smartband controller 220 or smart scale controller 320 can further perform, based on the configurations, the bioimpedance measurements. The results of bioimpedance measurement can be kept in memory of the smartband 110 or smartband 120 or can be sent to the computing device 140.

In some embodiments, a raw data of bioimpedance measurements (for example, voltage differences on the voltage electrodes) corresponding to one or more of the configurations shown in the FIG. 4 can be provided to computing device 140. Computing device 140 can analyze the results of the bioimpedance measurements performed for the different configurations 405, 410, 415, 420, 425, and 430 to estimate body composition of a user of the smartband 110 and smart scale 120.

Prior to the bioimpedance measurements, the user can be advised, via an application on computing device 140, to connect smartband 110 and smart scale 120 with wire 130, to step on the smart scale 120, and to touch current electrode 260 and voltage electrode 265 on outer surface of smartband 110.

Configuration 405 can be used for segmental hand-to-hand bioimpedance measurements performed using current electrodes 250 and 260 and voltage electrodes 255 and 265 of smartband 110. Electrical current is provided to current electrodes 250 and 260 and voltage difference is measured from voltage electrodes 255 and 265.

Configuration 410 can be used for segmental leg-to-leg bioimpedance measurements performed using current electrodes 350 and 360 and voltage electrodes 355 and 365 of smart scales 120. Electrical current is provided to current electrodes 350 and 360 and voltage difference is measured from voltage electrodes 355 and 365.

Configuration 415 can be used for whole-body left-hand-to-right-leg bioimpedance measurements performed using current electrode 260 and voltage electrode 265 of smartband 110 and current electrode 350 and voltage electrode 355 of smart scale 120. Electrical current is provided to current electrodes 260 and 350 and voltage difference is measured from voltage electrodes 265 and 355.

Configuration 420 can be used for whole-body right-hand-to-right-leg bioimpedance measurements performed using current electrode 250 and voltage electrode 255 of smartband 110 and current electrode 350 and voltage electrode 355 of smart scale 120. Electrical current is provided to current electrodes 250 and 350 and voltage difference is measured from voltage electrodes 255 and 355.

Configuration 425 can be used for whole-body hand-to-legs bioimpedance measurements performed using current electrodes 250 and 260 of smartband 110 and voltage electrodes 355 and 365 of smart scale 120. Electrical current is provided to current electrodes 250 and 260 and voltage difference is measured from voltage electrodes 355 and 365.

Configuration 430 can be used for integrated segmental body bioimpedance measurements performed using current electrodes 250 and 260 and voltage electrode 255 and 265 of smartband 110 and current electrodes 350 and 360 and voltage electrode 355 and 365 of smart scale 120. Electrical current is provided to all the current electrodes 250, 260, 350, and 360. In configuration 430, four voltage differences can be measured: from voltage electrodes 255 and 265, from voltage electrodes 355 and 365, from voltage electrodes 255 and 365, and from voltage electrodes 265 and 355.

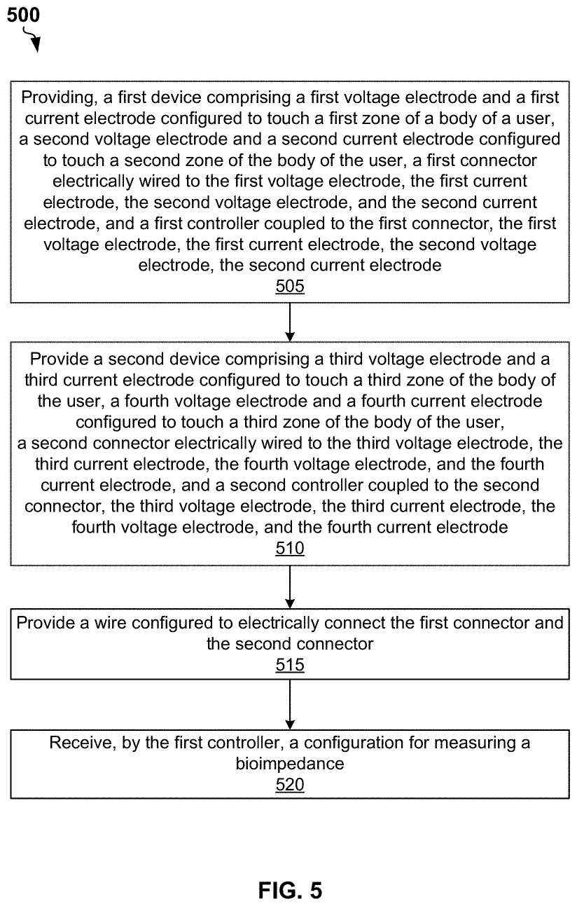

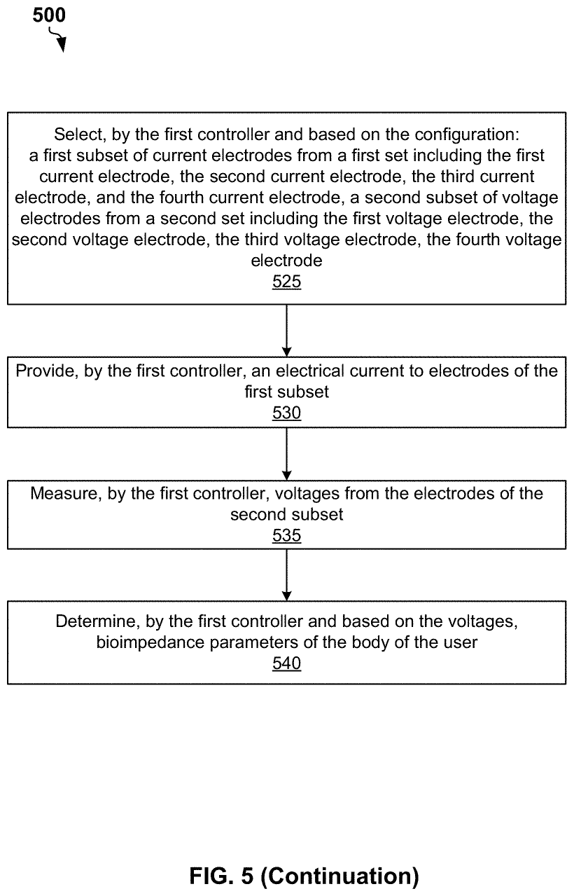

FIG. 5 is a flow chart of a method 500 for combined inter-device multi-point bioimpedance measurements, according to some example embodiments. The method 500 may commence in block 505 with providing a first device. The first device includes a first voltage electrode and a first current electrode configured to touch a first zone of a body of a user. The first device includes a second voltage electrode and a second current electrode configured to touch a second zone of the body of the user. The first device may include a first connector electrically wired to the first voltage electrode, the first current electrode, the second voltage electrode, and the second current electrode. The first device includes a first controller coupled to the first connector, the first voltage electrode, the first current electrode, the second voltage electrode, and the second current electrode.

In block 510, the method 500 may include providing a second device. The second device includes a third voltage electrode and a third current electrode configured to touch a third zone of the body of the user. The second device includes a fourth voltage electrode and a fourth current electrode configured to touch a third zone of the body of the user. The second device includes a second connector electrically wired to the third voltage electrode, the third current electrode, the fourth voltage electrode, and the fourth current electrode. The second device includes a second controller coupled to the second connector, the third voltage electrode, the third current electrode, the fourth voltage electrode, and the fourth current electrode.

The first device can be a smartband. The first voltage electrode and the first current electrode can be configured to touch the skin of the first hand of the user. The second voltage electrode and the second current electrode can be configured to be touched by the skin of the second hand of the user.

The second device can be a smart scale. The third voltage electrode and the third current electrode can be configured to touch the skin of the first foot of the user. The fourth voltage electrode and the fourth current electrode can be configured to touch the skin of the second food of the user.

In block 515, the method 500 may include providing a wire configured to electrically connect the first connector and the second connector.

In block 520, the method 500 may include receiving, by the first controller, a configuration for measuring bioimpedance.

In block 525, the method 500 may include selecting, by the first controller and based on the configuration, a first subset of current electrodes from a first set including the first current electrode, the second current electrode, the third current electrode, and the fourth current electrode and a second subset of voltage electrodes from a second set including the first voltage electrode, the second voltage electrode, the third voltage electrode, and the fourth voltage electrode.

In one embodiment, the first subset includes at least one current electrode of the first device and the second subset includes at least one voltage electrode of the second device. Alternatively, the first subset includes at least one voltage electrode of the first device and the second subset includes at least one current electrode of the second device.

In block 530, the method 500 may include providing, by the first controller, an electrical current to electrodes of the first subset.

In block 535, the method 500 may include measuring, by the first controller, voltages from the electrodes of the second subset.

In one embodiment, providing electrical current is restricted to at least one of the first current electrode and the second current electrode and measuring of the voltages is restricted to at least one of the third voltage electrode and fourth voltage electrodes.

In another embodiment, providing electrical current is restricted to at least one of the third current electrode and the fourth current electrode and measuring of the voltages is restricted to at least one of the first voltage electrode and second voltage electrodes.

In block 540, the method 500 may include determining, by the first controller and based on the voltages, bioimpedance parameters of the body of the user.

In one embodiment, the first device includes a communication unit and the configuration is received, via the communication unit, from an external computational device. The method 500 may include sending, by the first controller, raw data of the measured voltages to the external computing device and analyzing, by the external computing device, the raw data to determine the bioimpedance parameters.

In one embodiment, the first device is configured to store first historical data of bioimpedance measured exclusively with the first voltage electrode, the first current electrode, the second voltage electrode, and the second current electrode and without connecting to the second device. The second device is configured to store second historical data of bioimpedance measured exclusively with the third voltage electrode, the third current electrode, the fourth voltage electrode, and the fourth current electrode without connecting to the first device. The method 500 may include, upon being connected with the wire, exchanging, between the first device and the second device, the first historical data and the second historical data.

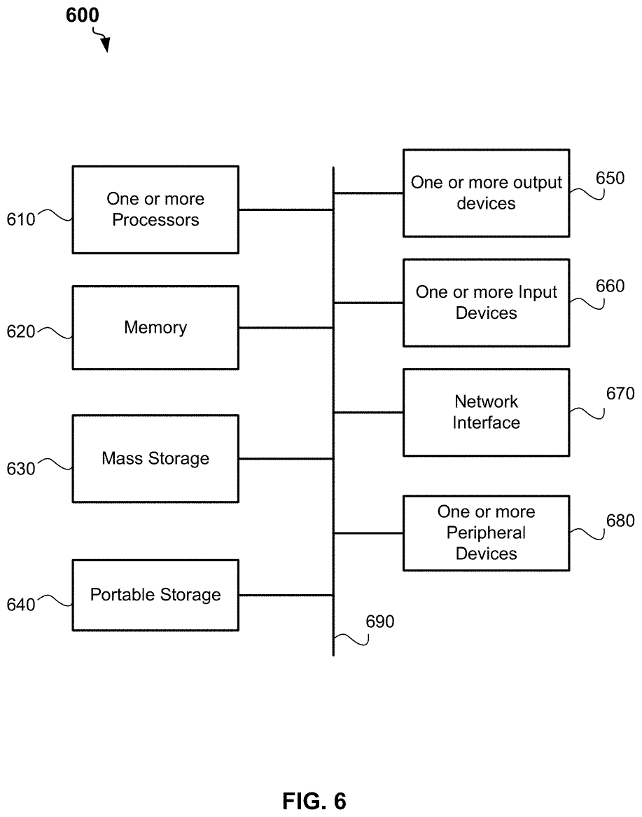

FIG. 6 illustrates an exemplary computing system 600 that may be used to implement embodiments described herein. Specifically, the computing system 600 can provide details of the computing device 140 shown in FIG. 1. The exemplary computing system 600 of FIG. 6 may include one or more processors 610 and memory 620. Memory 620 may store, in part, instructions and data for execution by the one or more processors 610. Memory 620 can store the executable code when the exemplary computing system 600 is in operation. The exemplary computing system 600 of FIG. 6 may further include a mass storage 630, portable storage 640, one or more output devices 650, one or more input devices 660, a network interface 670, and one or more peripheral devices 680.

The components shown in FIG. 6 are depicted as being connected via a single bus 690. The components may be connected through one or more data transport means. The one or more processors 610 and memory 620 may be connected via a local microprocessor bus, and the mass storage 630, one or more peripheral devices 680, portable storage 640, and network interface 670 may be connected via one or more input/output buses.

Mass storage 630, which may be implemented as a non-volatile storage device or other storage device for storing data and instructions, which may be used by one or more processors 610. Mass storage 630 can store the system software for implementing embodiments described herein for purposes of loading that software into memory 620.

Portable storage 640 may operate in conjunction with a portable non-volatile storage medium to input and output data and code to and from the computing system 600 of FIG. 6. The system software for implementing embodiments described herein may be stored on such a portable medium and input to the computing system 600 via the portable storage 640.

One or more input devices 660 provide a portion of a user interface. The one or more input devices 660 may include an alphanumeric keypad, such as a keyboard, for inputting alphanumeric and other information, or a pointing device, such as a mouse, a trackball, a stylus, or cursor direction keys. Additionally, the computing system 600 as shown in FIG. 6 includes one or more output devices 650. Suitable one or more output devices 650 include speakers, printers, network interfaces, and monitors.

Network interface 670 can be utilized to communicate with external devices, external computing devices, servers, and networked systems via one or more communications networks such as one or more wired, wireless, or optical networks including, for example, the Internet, intranet, LAN, WAN, cellular phone networks (e.g., Global System for Mobile communications network, packet switching communications network, circuit switching communications network), Bluetooth radio, and an IEEE 802.11-based radio frequency network, among others. Network interface 670 may be a network interface card, such as an Ethernet card, optical transceiver, radio frequency transceiver, or any other type of device that can send and receive information. Other examples of such network interfaces may include Bluetooth®, 3G, 4G, and WiFi® radios in mobile computing devices as well as a USB.

One or more peripheral devices 680 may include any type of computer support device to add additional functionality to the computing system. The one or more peripheral devices 680 may include a modem or a router.

The components contained in the exemplary computing system 600 of FIG. 6 are those typically found in computing systems that may be suitable for use with embodiments described herein and are intended to represent a broad category of such computer components that are well known in the art. Thus, the exemplary computing system 600 of FIG. 6 can be a PC, handheld computing device, telephone, mobile computing device, workstation, server, minicomputer, mainframe computer, or any other computing device. The computer can also include different bus configurations, networked platforms, multi-processor platforms, and so forth. Various operating systems (OS) can be used including UNIX, Linux, Windows, Macintosh OS, Palm OS, and other suitable operating systems.

Some of the above-described functions may be composed of instructions that are stored on storage media (e.g., computer-readable medium). The instructions may be retrieved and executed by the processor. Some examples of storage media are memory devices, tapes, disks, and the like. The instructions are operational when executed by the processor to direct the processor to operate in accord with the example embodiments. Those skilled in the art are familiar with instructions, processor(s), and storage media.

It is noteworthy that any hardware platform suitable for performing the processing described herein is suitable for use with the example embodiments. The terms “computer-readable storage medium” and “computer-readable storage media” as used herein refer to any medium or media that participate in providing instructions to a CPU for execution. Such media can take many forms, including, but not limited to, non-volatile media, volatile media, and transmission media. Non-volatile media include, for example, optical or magnetic disks, such as a fixed disk. Volatile media include dynamic memory, such as Random-Access-Memory (RAM). Transmission media include coaxial cables, copper wire, and fiber optics, among others, including the wires that include one embodiment of a bus. Transmission media can also take the form of acoustic or light waves, such as those generated during radio frequency and infrared data communications. Common forms of computer-readable media include, for example, a floppy disk, a flexible disk, a hard disk, magnetic tape, any other magnetic medium, a compact disk read-only memory (CD-ROM), a digital versatile disk (DVD), any other optical medium, a RAM, a programmable read-only memory (PROM), an erasable programmable read-only memory (EPROM), an electrically erasable programmable read-only memory (EEPROM), a flash memory storage, any other memory chip, a carrier wave, or any other medium from which a computer can read.

Various forms of computer-readable media may be involved in carrying one or more sequences of one or more instructions to a CPU for execution. A bus carries the data to system RAM, from which a CPU retrieves and executes the instructions. The instructions received by system RAM can optionally be stored on a fixed disk either before or after execution by a CPU.

Thus, systems and methods for combined inter-device multi-point bioimpedance measurements are described. Although embodiments have been described with reference to specific exemplary embodiments, it will be evident that various modifications and changes can be made to these exemplary embodiments without departing from the broader spirit and scope of the present application. Accordingly, the specification and drawings are to be regarded in an illustrative rather than a restrictive sense.

1. A system for performing combined inter-device multi-point bioimpedance measurements, the system comprising:
a first device comprising:
a first voltage electrode and a first current electrode configured to touch a first zone of a body of a user;
a second voltage electrode and a second current electrode configured to touch a second zone of the body of the user;
a first connector electrically wired to the first voltage electrode, the first current electrode, the second voltage electrode, and the second current electrode; and
a first controller coupled to the first connector, the first voltage electrode, the first current electrode, the second voltage electrode, and the second current electrode; and
a second device comprising:
a third voltage electrode and a third current electrode configured to touch a third zone of the body of the user;
a fourth voltage electrode and a fourth current electrode configured to touch a fourth zone of the body of the user;
a second connector electrically wired to the third voltage electrode, the third current electrode, the fourth voltage electrode, and the fourth current electrode; and
a second controller coupled to the second connector, the third voltage electrode, the third current electrode, the fourth voltage electrode, and the fourth current electrode; and
a wire configured to removably electrically connect the first connector and the second connector; and wherein
when the first device and the second device are connected with the wire:
the first controller is configured to:
receive a configuration for measuring a bioimpedance; select, based on the configuration:
a first subset of current electrodes from a first set including the first current electrode, the second current electrode, the third current electrode, and the fourth current electrode; and
a second subset of voltage electrodes from a second set including the first voltage electrode, the second voltage electrode, the third voltage electrode, the fourth voltage electrode;
provide an electrical current to electrodes of the first subset; measure voltages from the electrodes of the second subset; and
determine based on the voltages, reference bioimpedance parameters indicative of results of leg-to-hand bioimpedance measurements and when the first device and the second device are disconnected:
the first device is configured to:
perform first bioimpedance measurements independently from the second device to obtain first bioimpedance parameters indicative of results of hand-to-hand bioimpedance measurements; and
analyze the first bioimpedance parameters and the reference bioimpedance parameters to obtain a first estimate of a total body composition of the user; and
the second device is configured to:
perform second bioimpedance measurements independently from the first device to obtain second bioimpedance parameters; and
analyze the second bioimpedance parameters and the reference bioimpedance parameters to obtain a second estimate of the total body composition of the user.

2. The system of claim 1, wherein:
the first device is a smartband;
the first voltage electrode and the first current electrode are configured to touch the skin of a first hand of the user; and
the second voltage electrode and the second current electrode are configured to be touched by the skin of a second hand of the user.

3. The system of claim 1, wherein:
the second device is a smart scale;
the third voltage electrode and the third current electrode configured to touch the skin of a first foot of the user; and
the fourth voltage electrode and the fourth current electrode are configured to touch the skin of a second foot of the user.

4. The system of claim 1, wherein one of:
the first subset includes at least one current electrode of the first device and the second subset includes at least one voltage electrode of the second device; or
the first subset includes at least one voltage electrode of the first device and the second subset includes at least one current electrode of the second device.

5. The system of claim 1, wherein:
the providing electrical current is restricted to at least one of the first current electrode and the second current electrode; and
the measuring of the voltages is restricted to at least one of the third voltage electrode and fourth voltage electrodes.

6. The system of claim 1, wherein one of:
the providing electrical current is restricted to at least one of the third current electrode and the fourth current electrode; and
the measuring of the voltages is restricted to at least one of the first voltage electrode and second voltage electrodes.

7. The system of claim 1, wherein:
the first device includes a communication unit; and
the first controller is configured to:
receive, via the communication unit, the configuration from an external computational device; and
send, via the communication unit, the reference bioimpedance parameters to the external computing device.

8. The system of claim 7, wherein:
the first controller is configured to send raw data of the measured voltages to the external computing device; and
the external computing device is configured to analyze the raw data to determine the reference bioimpedance parameters.

9. The system of claim 7, wherein the communication unit is a wireless communication unit.

10. The system of claim 1, wherein:
the first device is configured to store first historical data of bioimpedance measured exclusively with the first voltage electrode, the first current electrode, the second voltage electrode and the second current electrode, without connecting to the second device;
the second device is configured to store second historical data of bioimpedance measured exclusively with the third voltage electrode, the third current electrode, the fourth voltage electrode and the fourth current electrode and without connecting to the first device; and
upon being connected with the wire, the first device and the second device are configured to exchange the first historical data and the second historical data.

11. A method for performing combined inter-device multi-point bioimpedance measurements, the method comprising:
providing, a first device including:
a first voltage electrode and a first current electrode configured to touch a first zone of a body of a user;
a second voltage electrode and a second current electrode configured to touch a second zone of the body of the user;
a first connector electrically wired to the first voltage electrode, the first current electrode, the second voltage electrode, and the second current electrode;
a first controller coupled to the first connector, the first voltage electrode, the first current electrode, the second voltage electrode, and the second current electrode; and providing a second device comprising:
a third voltage electrode and a third current electrode configured to touch a third zone of the body of the user;
a fourth voltage electrode and a fourth current electrode configured to touch a fourth zone of the body of the user;
a second connector electrically wired to the third voltage electrode, the third current electrode, the fourth voltage electrode, and the fourth current electrode; and
a second controller coupled to the second connector, the third voltage electrode, the third current electrode, the fourth voltage electrode, and the fourth current electrode; and
providing a wire configured to removably electrically connect the first connector and the second connector;
when the first device and the second device are connected with the wire:
receiving, by the first controller, a configuration for measuring a bioimpedance;
selecting, by the first controller and based on the configuration:
a first subset of current electrodes from a first set including the first current electrode, the second current electrode, the third current electrode, and the fourth current electrode; and
a second subset of voltage electrodes from a second set including the first voltage electrode, the second voltage electrode, the third voltage electrode, and the fourth voltage electrode;
providing, by the first controller, an electrical current to electrodes of the first subset;
measuring, by the first controller, voltages from the electrodes of the second subset; and
determining, by the first controller and based on the voltages, reference bioimpedance parameters indicative of results of leg-to-hand bioimpedance measurements; and
when the first device and the second device are disconnected:
performing, by the first device, first bioimpedance measurements independently from the second device to obtain first bioimpedance parameters indicative of results of hand-to-hand bioimpedance measurements; and
analyzing, by the first device, the first bioimpedance parameters and the reference bioimpedance parameters to obtain a first estimate of a total body composition of the user; and
performing, by the second device, second bioimpedance measurements independently from the first device to obtain second bioimpedance parameters; and
analyzing, by the second device, the second bioimpedance parameters and the reference bioimpedance parameters to obtain a second estimate of the total body composition of the user.

12. The method of claim 11, wherein:
the first device is a smartband;
the first voltage electrode and the first current electrode are configured to touch the skin of a first hand of the user; and
the second voltage electrode and the second current electrode are configured to be touched by the skin of a second hand of the user.

13. The method of claim 11, wherein:
the second device is a smart scale;
the third voltage electrode and the third current electrode configured to touch the skin of a first foot of the user; and
the fourth voltage electrode and the fourth current electrode are configured to touch the skin of a second food of the user.

14. The method of claim 11, wherein one of:
the first subset includes at least one current electrode of the first device and the second subset includes at least one voltage electrode of the second device; or
the first subset includes at least one voltage electrode of the first device and the second subset includes at least one current electrode of the second device.

15. The method of claim 11, wherein:
the providing electrical current is restricted to at least one of the first current electrode and the second current electrode; and
the measuring of the voltages is restricted to at least one of the third voltage electrode and fourth voltage electrodes.

16. The method of claim 11, wherein one of:
the providing electrical current is restricted to at least one of the third current electrode and the fourth current electrode; and
the measuring of the voltages is restricted to at least one of the first voltage electrode and second voltage electrodes.

17. The method of claim 11, wherein:
the first device includes a communication unit; and
the configuration is received, via the communication unit, from an external computational device.

18. The method of claim 17, further comprising:
sending, by the first controller, raw data of the measured voltages to the external computing device; and
analyzing, by the external computing device, the raw data to determine the reference bioimpedance parameters.

19. The method of claim 11, wherein:
the first device is configured to store first historical data of bioimpedance measured exclusively with the first voltage electrode, the first current electrode, the second voltage electrode, and the second current electrode and without connecting to the second device;
the second device is configured to store second historical data of bioimpedance measured exclusively with the third voltage electrode, the third current electrode, the fourth voltage electrode, and the fourth current electrode without connecting to the first device; and further comprising:
upon connecting with the wire, exchanging, between the first device and the second device, the first historical data and the second historical data.

20. A system for performing combined inter-device multi-point bioimpedance measurements, the system comprising:
a first device comprising:
a first voltage electrode and a first current electrode configured to touch a first zone of a body of a user;
a second voltage electrode and a second current electrode configured to touch a second zone of the body of the user;
a first connector electrically wired to the first voltage electrode, the first current electrode, the second voltage electrode, and the second current electrode; and
a first controller coupled to the first connector, the first voltage electrode, the first current electrode, the second voltage electrode, and the second current electrode; and
a second device comprising:
a third voltage electrode and a third current electrode configured to touch a third zone of the body of the user;
a fourth voltage electrode and a fourth current electrode configured to touch a fourth zone of the body of the user;
a second connector electrically wired to the third voltage electrode, the third current electrode, the fourth voltage electrode, and the fourth current electrode;
a second controller coupled to the second connector, the third voltage electrode, the third current electrode, the fourth voltage electrode, and the fourth current electrode; and
a wire configured to removably electrically connect the first connector and the second connector; and
an external computing device communicatively connected to the first device and the second device; and wherein
when the first device and the second device are connected with the wire:
at least one of the first controller and the second controller is configured to:
receive, from the external computing device, a configuration for measuring bioimpedance;
select, based on the configuration:
a first subset of current electrodes from a first set including the first current electrode, the second current electrode, the third current electrode, and the fourth current electrode; and
a second subset of voltage electrodes from a second set including the first voltage electrode, the second voltage electrode, the third voltage electrode, and the fourth voltage electrode;
provide an electrical current to electrodes of the first subset;
measure voltages from the electrodes of the second subset;
determine based on the voltages, reference bioimpedance parameters indicative of results of leg-to-hand bioimpedance measurements; and
send the reference bioimpedance parameters to the external computing device and
when the first device and the second device are disconnected:
the first device is configured to:
perform first bioimpedance measurements independently from the second device to obtain first bioimpedance parameters indicative of results of hand-to-hand bioimpedance measurements; and
analyze the first bioimpedance parameters and the reference bioimpedance parameters to obtain a first estimate of a total body composition of the user; and
the second device is configured to:
perform second bioimpedance measurements independently from the first device to obtain second bioimpedance parameters; and
analyze the second bioimpedance parameters and the reference bioimpedance parameters to obtain a second estimate of the total body composition of the user.
